(19) United States
(12) Patent Application Publication
(10) Pub. No.: US 2024/0278235 A1
(43) Pub. Date: Aug. 22, 2024

(54) AUTONOMOUS ROBOTIC SYSTEM FOR BIOCHEMICAL TESTING IN HEALTHCARE ENVIRONMENTS

(71) Applicant: ACCESS MEDICAL SYSTEMS, LTD., Palo Alto, CA (US)

(72) Inventor: Hong Tan, San Jose, CA (US)

(21) Appl. No.: 18/652,649

(22) Filed: May 1, 2024

Related U.S. Application Data
(63) Continuation of application No. PCT/US2022/079246, filed on Nov. 3, 2022.
(60) Provisional application No. 63/275,545, filed on Nov. 4, 2021.

(51) Int. Cl. B01L 3/00; G01N 35/04; G16H 50/20

(52) U.S. Cl. CPC B01L 3/5025; G16H 50/20; G01N 2035/0429

(57) ABSTRACT

Introduced here are diagnostic systems that are able to autonomously move to a desired location within a healthcare environment. As such, a diagnostic system may allow an operator to conduct a biochemical test on site. A diagnostic system may include (i) a cartridge in which a patient sample can be deposited, (ii) a robotic cart in which the cartridge can be inserted for analysis, and (iii) a control platform that is responsible for managing movement of the robotic cart throughout its physical environment. In addition to having the components needed to analyze the sample deposited into the cartridge, the robotic cart could also include storage for “used” or “unused” cartridges. Thus, the robotic cart may not only be able to perform analysis of the sample on site, but the robotic cart may also be able to transport all of the supplies needed to obtain the sample to the operator.

### CROSS-REFERENCE TO RELATED APPLICATIONS

This application is a continuation of International Application No. PCT/US2022/079246, filed on Nov. 3, 2022, which claims priority to U.S. Provisional Application No. 63/275,545, filed on Nov. 4, 2021, each of which is incorporated by reference herein in its entirety.

### FIELD OF THE INVENTION

Various embodiments concern mobile robotic devices that are able to autonomously traverse physical environments to perform biochemical testing.

### BACKGROUND

Mobile robotic devices (also referred to as “mobile robots”) are electronic machines that are able to move around physical environments, and therefore are not fixed to a single physical location. Many mobile robots rely on instructions received from guidance devices (also referred to as “controllers”) to navigate uncontrolled physical spaces. For example, a mobile robot may rely on instructions received from a corresponding controller to travel a predefined navigation route in a relatively controlled space. However, some mobile robots are capable of navigating uncontrolled physical environments without the need for controllers. Said another way, these mobile robots can operate autonomously with minimal oversight from an individual (also referred to as an “operator”). Operators will normally be healthcare professionals, though that need not necessarily be the case.

Autonomous robotics is generally considered to involve a combination of interdisciplinary fields, including artificial intelligence, robotics, and information engineering. While the term “autonomous” could be used to indicate that a mobile robot is able to perform any task with a high degree of autonomy (i.e., without external influence), that term is generally used to refer to the ability of the mobile robot to navigate a physical space with little or no assistance. To successfully navigate a physical space, a mobile robot must know its own position—a concept referred to as “localization”—and then be able to navigate around obstacles in the physical space.

Mobile robots have been tasked to perform various tasks in various physical environments. For example, mobile robots have been used to autonomously deliver goods from a source to a destination. As another example, mobile robots have been used to surveil areas that are impractical, inappropriate, or impossible for individuals to visit. There are several physical environments that have historically been ill suited for mobile robots, however. One of these physical environments is healthcare facilities. Although several entities have developed mobile robots for healthcare facilities, adoption has been limited in part because the usefulness of these mobile robots is limited.

### BRIEF DESCRIPTION OF THE DRAWINGS

FIG. 1 depicts a biosensor interferometer that includes a light source, a detector, a waveguide, and an optical assembly.

FIG. 2 depicts an example of a probe in accordance with various embodiments.

FIGS. 3A-B illustrate the principles of detection in a thin-film interferometer.

FIG. 4 depicts an example of a slide in accordance with various embodiments.

FIG. 5 depicts another example of a slide in accordance with various embodiments.

FIG. 6 depicts an example of a cartridge assembly in accordance with various embodiments.

FIG. 7A includes a perspective view of a robotic cart in accordance with various embodiments.

FIG. 7B includes a block diagram illustrating various components of the robotic cart shown in FIG. 7A.

FIG. 8 is a high-level block diagram illustrating how a robotic cart may be able to interface with a hub unit, for example, for charging purposes.

FIG. 9 includes a high-level illustration of communications that may occur between a control platform, a robotic cart, and a hub unit.

FIG. 10 illustrates a network environment that includes a control platform.

FIG. 11 is a block diagram illustrating an example of a processing system in which at least some operations described herein can be implemented.

Embodiments are illustrated by way of example and not limitation in the drawings. While the drawings depict various embodiments for the purpose of illustration, those skilled in the art will recognize that alternative embodiments may be employed without departing from the principles of the technology. Accordingly, while specific embodiments are shown in the drawings, the technology is amenable to various modifications.

### DETAILED DESCRIPTION

Biochemical tests based on binding events between analyze molecules and analyte-binding molecules are widely used in medical, veterinary, agricultural, and research applications. These biochemical tests can be employed to detect whether analyte molecules are present in a sample, the amount of analyte molecules in a sample, or the rate at which analyte molecules bind to analyte-binding molecules. Together, an analyte-binding molecule and its corresponding analyte molecule form an analyte-anti-analyte binding pair (or simply “binding pair”). Examples of binding pairs include complementary strands of nucleic acids, antigen-antibody pairs, and receptor-receptor binding agents. The analyte can be either member of the binding pair, and the anti-analyte can be the other member of the binding pair.

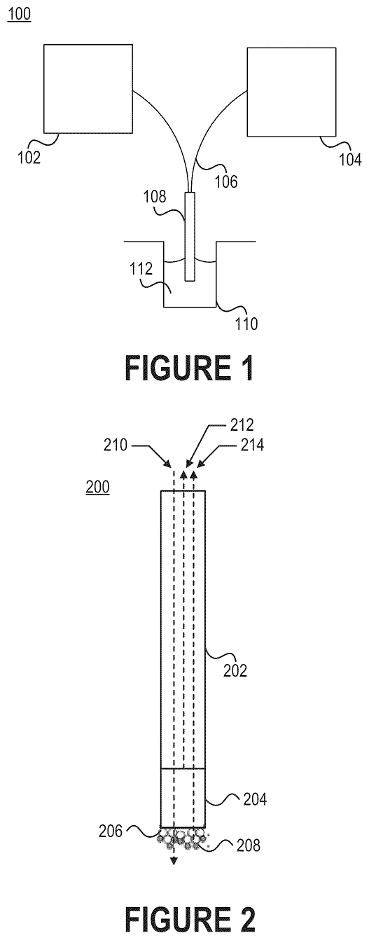

Several entities have developed systems that are designed to conduct biochemical tests. FIG. 1 illustrates an example of a system that is able to monitor adherence of analyte molecules to analyte-binding molecules immobilized on a surface, while FIGS. 2, 4, and 5 illustrate different examples of surfaces on which analyte-binding molecules can be immobilized. These drawings are discussed in greater detail below.

These biochemical tests have several drawbacks, however. One drawback is the resources that are needed to conduct a biochemical test. Assume, for example, that a sample (e.g., of blood) associated with a patient is to be biochemically tested to determine whether a given analyte is present in the sample. In such a scenario, a healthcare professional may need to obtain the sample and then traverse a healthcare environment to find a cartridge that includes at least one interferometric sensor and one or more wells in which fluids can be deposited. Thereafter, the healthcare professional may deposit at least a portion of the sample in a well and then insert the cartridge into a system that is able to examine the sample. Outputs produced by the system may be analyzed by the healthcare professional, who might then return to the patient to describe the outputs. For example, the healthcare professional may provide a diagnosis for an ailment if the system determines that an analyte indicative of the ailment is present in the sample.

Although the system may be located in the same healthcare environment as the patient, the system is rarely, if ever, proximate to the patient. For example, the patient may be situated in a room upon being admitted to a hospital, while the system may be situated in a laboratory that is located in another room, floor, or building altogether. Accordingly, the process for conducting biochemical tests may not only be laborious, but also expensive due to the time that healthcare professionals must spend on these biochemical tests. Moving samples from one location (e.g., the room assigned to a patient) to another location (e.g., a laboratory) also introduces another opportunity for these samples to be lost, tainted, or otherwise mismanaged.

Introduced here is a diagnostic system that is able to autonomously move to a desired location within a healthcare environment. As such, the diagnostic system may allow an operator (also referred to as a “user”) to conduct a biochemical test on site. For example, the operator may be able to conduct a biochemical test from alongside the bed in which a patient is situated.

As further discussed below, the diagnostic system may include (i) a cartridge in which a sample can be deposited, (ii) a robotic cart in which the cartridge can be inserted for analysis, and (iii) a control platform that is responsible for managing movement of the robotic cart throughout its physical environment. In addition to having the components needed to analyze the sample deposited into the cartridge, the robotic cart could also include storage for “used” or “unused” cartridges. Thus, the robotic cart may not only be able to perform analysis of the sample on site, but the robotic cart may also be able to transport all of the supplies needed to obtain the sample to the operator.

Assume, for example, that an operator is interested in conducting a biochemical test to determine whether a given analyte is present in a sample. In such a scenario, the operator may provide, to the control platform, input indicative of a request to summon the robotic cart. As further discussed below, the control platform may be embodied as a computer program (e.g., a mobile application) that is executing on a computing device (e.g., a mobile phone) that the operator is able to access. Upon receiving this input, the control platform may transmit an instruction to the robotic cart to locate itself proximate to the operator. In some embodiments the operator may be prompted to specify her location (e.g., through the computer program), while in other embodiments the location of the operator may be inferred by the control platform (e.g., based on a location of the computing device, a schedule of the operator, etc.).

After the robotic cart arrives at its intended destination, the operator may open the storage and remove a cartridge. Normally, ancillary materials like syringes and needles will already be readily accessible, through the robotic cart could also store these materials. The operator can obtain a sample and deposit the sample into the cartridge, and then the operator can load the cartridge into the robotic cart for analysis. The outputs that are produced by the robotic cart can be presented by the robotic cart (e.g., on a display). Additionally or alternatively, the outputs that are produced by the robotic cart can be forwarded to the control platform for presentation on the computing device.

Such an approach to locally performing biochemical tests will not only save costs by lessening the amount of time that these tests require, but can also ensure prompt results as the analysis can be performed by the robotic cart without requiring that the sample be transported to another location. Lessening the distance over which samples must be transported may also help reduce errors that are caused by tainting or otherwise mismanaging samples.

Note that while the robotic cart may be described as having the necessary equipment for conducting biochemical tests using interferometric sensors, the robotic cart could additionally or alternatively include the equipment needed to conduct other types of biochemical tests. Accordingly, the robotic cart may be able to facilitate interferometric assays, fluorescent assays, chemiluminescent-fluorescent assays, or combinations thereof. Fluorescent assays are further described in U.S. Pat. No. 8,647,889, titled “Luminescent Polymer Cyclic Amplification,” while chemiluminescent-fluorescent assays are further described in U.S. Provisional Application No. 63/129,946, titled “Detection Method Using Both Fluorescence and Chemiluminescence Labels,” each of which is incorporated by reference herein in its entirety. Meanwhile, interferometric assays are further described in International Application No. PCT/US2021/021653, titled “Interferometric Sensors for Biochemical Testing,” which is also incorporated by reference herein in its entirety.

### Definitions

The term “about” means within ±10% of the recited value.

The term “analyte-binding molecule” refers to any molecule capable of participating in a binding reaction with an analyte molecule. Examples of analyte-binding molecules include, but are not limited to, (i) antigen molecules; (ii) antibody molecules; (iii) protein molecules; (iv) ligands; and (v) single-stranded nucleic acid molecules.

The term “interferometric sensor” refers to any sensing apparatus upon which a biolayer formed to produce an interference pattern. One example of an interferometric sensor is a probe designed to be suspended in a solution containing the sample having the analyte molecules. Another example of an interferometric sensor is a slide with a planar surface upon which a biolayer can be formed over the course of a biochemical test.

The term “probe” refers to a monolithic substrate having as aspect ratio (length-to-width) of at least 2 to 1 with a thin-film layer coated on the sensing side.

The term “monolithic substrate” refers to a solid piece of material having a uniform composition, such as glass, quartz, or plastic, with one refractive index.

The term “waveguide” refers to a device (e.g., a duct, coaxial cable, or optic fiber) designed to confine and direct the propagation of electromagnetic waves (as light). One example of a waveguide is a metal tube for channeling ultrahigh-frequency waves.

The term “healthcare professional” may refer to any individual who is involved in conducting a biochemical test. This term is commonly used to refer to physicians and nurses, though this term could also be used to refer to researchers and other individuals who are not directly involved in administering health care to patients.

The term “healthcare environment” may refer to a physical space in which health care is provided to patients. Examples of healthcare environments include hospitals, clinics, nursing homes, and hospice facilities.

### Interferometric Approach to Biochemical Testing

FIG. 1 depicts a biosensor interferometer 100 (or simply “interferometer”) that includes a light source 102, a detector 104, a waveguide 106, and an optical assembly 108 (also referred to as a “probe”). The probe 108 may be connected to the waveguide 106 via a coupling medium.

The light source 102 may emit light that is guided toward the probe 108 by the waveguide 106. For example, the light source 102 may be a light-emitting diode (LED) that is configured to produce light over a range of at least 50 nanometers (nm), 100 nm, or 150 nm within a given spectrum (e.g., 400 nm or less to 700 nm or greater). Alternatively, the interferometer 100 may employ a plurality of light sources having different characteristic wavelengths, such as LEDs designed to emit light at different wavelengths in the visible range. The same function could be achieved by a single light source with suitable filters for directing light with different wavelengths onto the probe 108.

The detector 104 is preferably a spectrometer that is capable of recording the spectrum of interfering light received from the probe 108. Alternatively, if the light source 102 operates to direct different wavelengths onto the probe 108, then the detector 104 can be a simple photodetector capable of recording intensity at each wavelength. In another embodiment, the detector 104 can include multiple filters that permit detection of intensity at each of multiple wavelengths.

The waveguide 106 can be configured to transport light emitted by the light source 102 to the probe 108, and then transport light reflected by surfaces within the probe 108 to the detector 104. In some embodiments the waveguide 106 is a bundle of optical fibers (e.g., single-mode fiber optic cables), while in other embodiments the waveguide 106 is a multi-mode fiber optic cable.

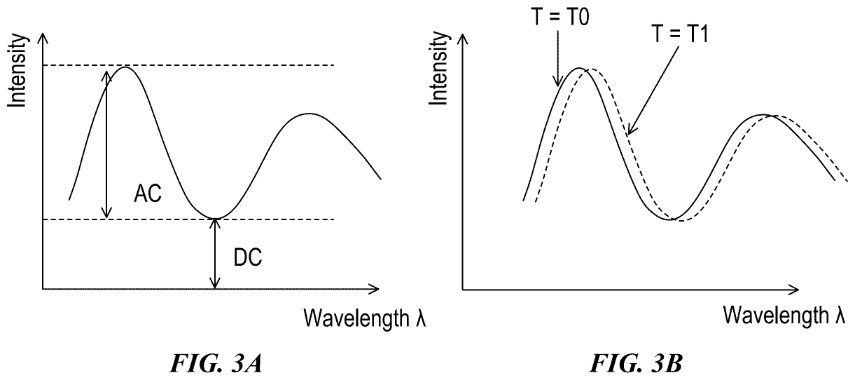

As shown in FIGS. 2-3, the probe 108 can include a monolithic substrate, a thin-film layer (also referred to as an “interference layer”), and a biomolecular layer (also referred to as a “biolayer”) that comprises analyte molecules that have bound to analyte-binding molecules. The monolithic substrate comprises a transparent material through which light can travel. The interference layer also comprises a transparent material. When light is shone on the probe 108, the proximal surface of the interference layer may act as a first reflecting surface and the biolayer may act as a second reflecting surface. As further described below, light reflected by the first and second reflecting surfaces may form an interference pattern that can be monitored by the interferometer 100.

To perform a diagnostic test, the probe 108 can be suspended in a microwell 110 (or simply “well”) that includes a sample 112. This well 110 may be formed in a cartridge as further discussed below. Analyte molecules in the sample 112 will bind to the analyte-binding molecules along the distal end of the probe 108 over the course of the diagnostic test, and these binding events will result in an interference pattern that can be observed by the detector 104. The interferometer 100 can monitor the thickness of the biolayer that is formed along the distal end of the probe 108 by detecting shifts in a phase characteristic of the interference pattern.

Note that, for the purpose of illustration, embodiments may be described in the context of a robotic cart that has an interferometer stored therein. However, the robotic cart could include other investigative tools for conducting biochemical tests as mentioned above. As an example, a robotic cart may be able to determine the presence or concentration of molecules through the use of fluorescent tags (also referred to as “fluorescent labels”) and/or chemiluminescence tags (also referred to as “chemiluminescence labels”). Accordingly, the robotic cart may simply be said to include an “analyzer” or “test system” that is able to administer, conduct, manage, or otherwise support biochemical tests. The “analyzer” may be able to facilitate interferometric assays, fluorescent assays, chemiluminescent assays, combination assays (e.g., chemiluminescent-fluorescent assays), or any combination thereof.

### Overview of Interferometric Sensors

The probe that is suspended in the well may be representative of an interferometric sensor (also referred to as an “interferometric biosensor” or “sensing apparatus”). Generally, the probe includes a monolithic substrate that has first and second surfaces arranged substantially parallel to one another at opposite ends of the monolithic substrate, an interference layer coated on the second surface of the monolithic substrate, and a layer of analyte-binding molecules coated on the interference layer. A first interface between the monolithic substrate and the interference layer acts as a first reflecting surface when light is shone on the probe, while a second interface between a biolayer formed by analyte molecules in a sample binding to the analyte-binding molecules and a solution containing the sample acts as a second reflecting surface when the light is shone on the probe. As described above, the thickness of the biolayer can be estimated based on the interference pattern of light reflected by the first and second reflecting surfaces.

FIG. 2 depicts an example of a probe 200 in accordance with various embodiments. The probe 200 includes an interference layer 204 that is secured along the distal end of a monolithic substrate 202. Analyte-binding molecules 206 can be deposited along the distal surface of the interference layer 204. Over the course of a biochemical test, a biolayer will form as analyte molecules 208 in a sample bind to the analyte-binding molecules 206.

As shown in FIG. 2, the monolithic substrate 202 has a proximal surface (also referred to as a “coupling side”) that can be coupled to, for example, a waveguide of an interferometer and a distal surface (also referred to as a “sensing side”) on which additional layers are deposited. Generally, the monolithic substrate 202 has a length of at least 3 millimeters (mm), 5 mm, 10 mm, or 15 mm. In a preferred embodiment, the aspect ratio (length-to-width) of the monolithic substrate 202 is at least 5 to 1. In such embodiments, the monolithic substrate 202 may be said to have a columnar form. The cross section of the monolithic substrate 202 may a circle, oval, square, rectangle, triangle, pentagon, etc. The monolithic substrate 202 preferably has a refractive index that is substantially higher than the refractive index of the interference layer 204, such that the proximal surface of the interference layer 204 effectively reflects light directed onto the probe 200. The preferred refractive index of the monolithic substrate may be higher than 1.5, 1.8, or 2.0. Accordingly, the monolithic substrate 202 may comprise a high-refractive-index material such as glass (refractive index of 2.0), though some embodiments of the monolithic substrate 202 may comprise a low-refractive-index material such as quartz (refractive index of 1.46) or plastic (refractive index of 1.32-1.49).

The interference layer 204 comprises at least one transparent material that is coated on the distal surface of the monolithic substrate 202. These transparent material(s) are deposited on the distal surface of the monolithic substrate 202 in the form of thin films ranging in thickness from fractions of a nanometer (e.g., a monolayer) to several micrometers. The interference layer 204 may have a thickness of at least 500 nm, 700 nm, or 900 nm. An exemplary thickness is between 500-5,000 nm (and preferably 800-1,200 nm). Here, for example, the interference layer 204 has a thickness of approximately 900-1,000 nm, or 940 nm.

Note that the interference layer 204 may have a substantially similar refractive index as the biolayer. This ensures that the reflection from the distal end of the probe 200 is predominantly due to the analyte molecules 208 rather than the interface between the interference layer 204 and the analyte-binding molecules 206. Generally, the biolayer has a refractive index of approximately 1.36, though this may vary depending on the type of analyte-binding molecules (and thus analyte molecules) along the distal end of the probe 200.

In some embodiments the interference layer 204 comprises magnesium fluoride (MgF₂), while in other embodiments the interference layer 204 comprises potassium fluoride (KF) with a refractive index of 1.36, lithium fluoride (LiF) with a refractive index of 1.39, sodium fluoride (NaF) with a refractive index of 1.32, lithium calcium aluminum fluoride (LiCaAlF₆) with a refractive index of 1.39, strontium fluoride (SrF₂) with a refractive index of 1.37, aluminum fluoride (AlF₃) with a refractive index of 1.38, sulphur hexafluoride (SF₆) with a refractive index of 1.00, sodium aluminum hexafluoride (Na₃AlF₆) (also referred to as “cryolite”) with a refractive index of 1.34, sodium aluminum fluoride (Na₅Al₃F₁₄) (also referred to as “chiolite”) with a refractive index of 1.34, etc. Additionally or alternatively, the interference layer 204 could comprise a polymer such as FICOLL®. Magnesium fluoride has a refractive index of 1.38, which is substantially identical to the refractive index of the biolayer formed along the distal end of the probe 200. Generally, the refractive index of the interference layer 204 is between 1.32 and 1.42, between 1.36 and 1.42, or between 1.38 and 1.40. Because the interference layer 204 and biolayer have similar refractive indexes, light will experience minimal scattering as it travels from the interference layer 204 into the biolayer and then returns from the biolayer into the interference layer 204.

The thickness of the biolayer is designed to optimize the overall sensitivity based on the hardware (e.g., the optical components) of the interferometer. Conventional immobilization chemistries can be used to covalently (e.g., chemically) or non-covalently (e.g., by adsorption) attach the analyte-binding molecules 206 to the distal surface of the interference layer 204.

The layer of analyte-binding molecules 206 is preferably formed under conditions in which the distal end of the probe 200 is densely coated, so that binding of analyte molecules 208 to the analyte-binding molecules 206 results in a change in the thickness of the biolayer rather than filling in the layer. The layer of analyte-binding molecules 206 can be a monolayer or a multi-layer matrix.

During a biochemical test, the probe 200 can be suspended within a cavity (e.g., a well) that includes a sample as shown in FIG. 1. Over the course of the biochemical test, a biolayer will form along the distal end of the probe 200 as analyte molecules 208 bind to the analyte-binding molecules 206.

When light is shone on the probe 200, the proximal surface of the interference layer 204 may act as a first reflecting surface and the distal surface of the biolayer may act as a second reflecting surface. The presence, concentration, or binding rate of analyte molecules 208 to the probe 200 can be estimated based on the interference of beams of light reflected by these two reflecting surfaces. As analyte molecules 208 attach to (or detach from) the analyte-binding molecules 206, the distance between the first and second reflecting surfaces will change. Because the dimensions of all other components in the probe 200 remain the same, the interference pattern formed by the light reflected by the first and second reflecting surfaces is phase shifted in accordance with changes in biolayer thickness due to binding events.

The use of a monolithic substrate 202 provides several advantages. As noted above, the refractive index of the monolithic substrate 202 is preferably higher than the refractive index of the interference layer 204. For example, the refractive index of the monolithic substrate 202 may be at least 0.1, 0.2, 0.4, 0.5, or 0.6 higher than the refractive index of the interference layer 204. Because the monolithic substrate 202 is a solid piece of material having a uniform composition, it is easier to select a material having a higher refractive index than that of the interference layer 204.

In operation, an incident light signal 210 emitted by a light source is transported through the monolithic substrate 202 toward the biolayer. Within the probe 200, light will be reflected at the first reflecting surface resulting in a first reflected light signal 212. Light will also be reflected at the second reflecting surface resulting in a second reflected light signal 214. The second reflecting surface initially corresponds to the interface between the analyte-binding molecules 206 and the sample in which the probe 200 is immersed. As binding occurs during the biochemical test, the second reflecting surface becomes the interface between the analyte molecules 208 and the sample.

The first and second reflected light signals 212, 214 form a spectral interference pattern, as shown in FIG. 3A. When analyte molecules 208 bind to the analyte-binding molecules 206 on the distal surface of the interference layer 204, the optical path of the second reflected light signal 214 will lengthen. As a result, the spectral interference pattern shifts from T0 to T1 as shown in FIG. 3B. By measuring the phase shift continuously in real time, a kinetic binding curve can be plotted as the amount of shift versus the time. The association rate of an analyte molecule to an analyte-binding molecule immobilized on the distal surface of the interference layer 204 can be used to calculate analyte concentration in the sample. Hence, the measure of the phase shift is the detection principle of a thin-film interferometer.

In some embodiments, a reflection layer (not shown) is deposited along the distal end of the monolithic substrate 202 such that the reflection layer is positioned between the monolithic substrate 202 and interference layer 204. Since its main purpose is to ensure that the first reflected light signal 212 reflects at the interface between the monolithic substrate 202 and interference layer 204, the reflection layer may comprise a material having a higher refractive index than either the monolithic substrate 202 or interference layer 204. The reflection layer may be very thin in comparison to the interference layer 204. For example, the reflection layer may have a thickness of approximately 3-10 nm.

Moreover, an adhesion layer (not shown) may be deposited along the distal surface of the interference layer 204 affixed to the monolithic substrate 202 in some embodiments. The adhesion layer may comprise a material that promotes adhesion of the analyte-binding molecules 206. One example of such a material is silicon dioxide. The adhesion layer is generally very thin in comparison to the interference layer 204, so its impact on light traveling toward, or returning from, the biolayer will be minimal. For example, the adhesion layer may have a thickness of approximately 3-10 nm, while the interference layer 204 may have a thickness of approximately 800-1,000 nm. The biolayer formed by the analyte-binding molecules 206 and analyte molecules 208 will normally have a thickness of several nm.

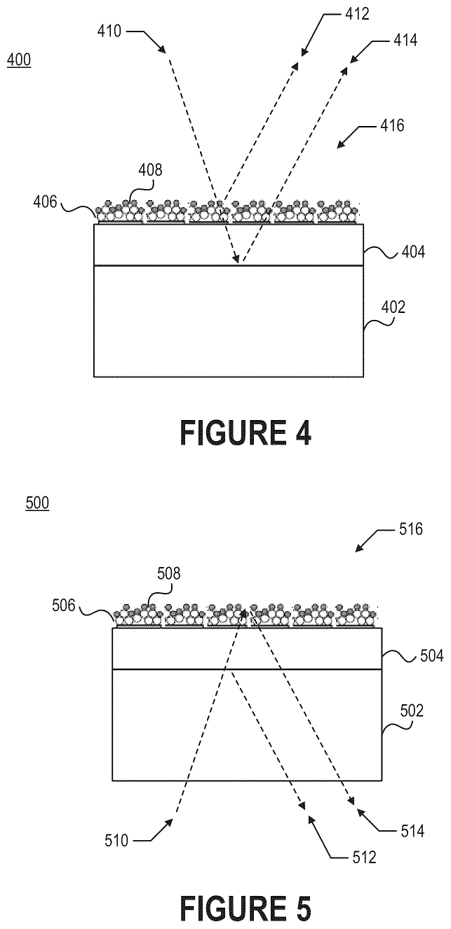

While embodiments may be described in the context of sensing surfaces having a columnar form, these features may be similarly applicable to sensing surfaces having other forms. One example of such a sensing surface is a slide (also referred to as a “chip”) with a planar surface upon which a biolayer is formed by flowing a solution over the planar surface over the course of a biochemical test. Several examples of planar surfaces are discussed below with reference to FIGS. 4-5.

FIG. 4 depicts an example of a slide 400 in accordance with various embodiments. The slide 400 includes a substrate 402 upon which an interference layer 404 is deposited. In some embodiments the interference layer 404 is deposited along the entire upper surface of the substrate 402, while in other embodiments the interference layer 404 is deposited along a portion of the upper surface of the substrate 402. For example, the interference layer 404 may be deposited within channels or wells formed within the upper surface of the substrate 402. As discussed above, the monolithic substrate 202 of FIG. 2 is generally much larger in height than in width. Here, however, the inverse may be true. In fact, the width of the substrate 402 may be larger than the length by a factor of 5, 7.5, 10, or 20. As an example, the substrate may be approximately 75 mm by 26 mm with a height/thickness of roughly 1 mm.

Over the course of a biochemical test, analyte molecules 408 can bind to analyte-binding molecules 406 that have been secured along the upper surface of the interference layer 404 to form a biolayer. To establish the thickness of the biolayer, light can be shone at the upper surface of the slide 400 as shown in FIG. 4. More specifically, an incident light signal 410 emitted by a light source can be shone at the biolayer that has formed along the upper surface of the slide 400. This may require that the incident light signal 410 travel through ambient media 416, which may be vacuum, air, or solution. The incident light signal 410 will be reflected at a first reflecting surface resulting in a first reflected light signal 412. The first reflecting surface may be representative of the interface between the biolayer and ambient media 416. The incident light signal 410 will also be reflected at a second reflecting surface resulting in a second reflected light signal 414. The second reflecting surface may be representative of the interface between the interference layer 404 and substrate 402. As discussed above, the first and second reflected light signals 412, 414 form a spectral interference pattern that can be analyzed to establish the thickness of the biolayer. Note that because the incident light signal 410 is not transported through the substrate 402, the substrate 402 could be either transparent or non-transparent (e.g., opaque).

FIG. 5 depicts another example of a slide 500 in accordance with various embodiments. Slide 500 of FIG. 5 may be largely similar to slide 400 of FIG. 4. Thus, the slide 500 may include a substrate 502 upon which an interference layer 504 and analyte-binding molecules 506 are deposited. Over the course of a diagnostic test, analyte molecules 508 can bind to the analyte-binding molecules 506 to form a biolayer.

Here, however, the incident light signal 510 is shone at the lower surface of the slide 500. In operation, the incident light signal 510 is transported through the substrate 502 toward the biolayer. Within the slide 500, light will be reflected at a first reflecting surface resulting in a first reflected light signal 512. The first reflecting surface may be representative of the interface between the interference layer 504 and substrate 502. Light will also be reflected at a second reflecting surface resulting in a second reflected light signal 514. The second reflecting surface may be representative of the interface between the biolayer and ambient media 516. As discussed above, the first and second reflected light signals 512, 514 form a spectral interference pattern that can be analyzed to establish the thickness of the biolayer.

While not shown in FIGS. 4-5, the slides 400, 500 could include a reflection layer that is disposed between the substrate 402, 502 and interference layer 404, 504 to improve reflectivity along that interface and/or an adhesion layer that is disposed along the upper surface of the interference layer 404, 504 to secure the analyze-binding molecules 406, 506.

### Overview of Cartridge

Regardless of its form, a sensing surface may need to contact a solution that contains a sample associated with a patient. For example, if the sensing surface is a probe with a columnar form as shown in FIG. 2, the sensing surface may be suspended in a well. This well may be formed along the surface of a cartridge that can be loaded into a robotic cart for analysis by an interferometer.

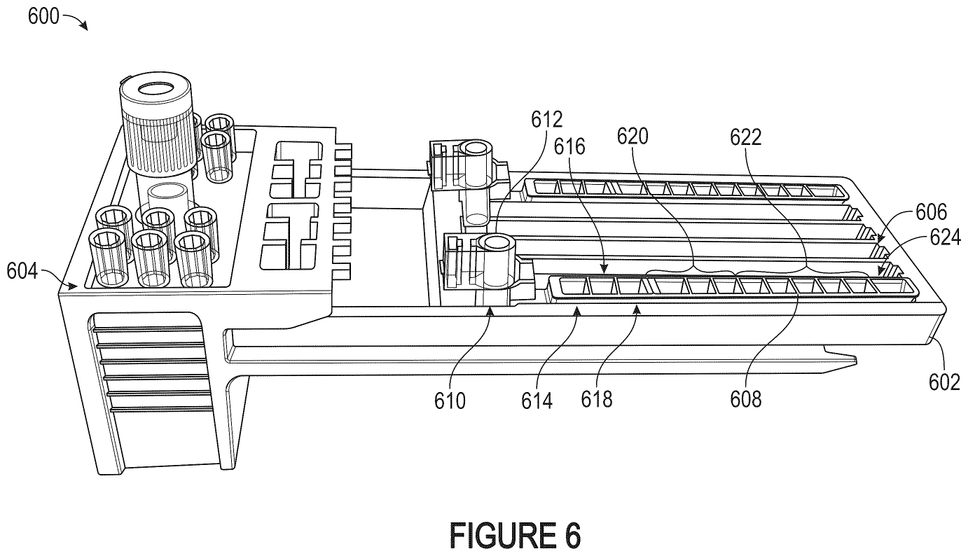

FIG. 6 depicts an example of a cartridge assembly 600 in accordance with various embodiments. The cartridge assembly 600 may include a tray 602 that includes a matrix of cavities 304 in which probes can be loaded, either temporarily (e.g., for cleaning) or semi-permanently (e.g., for storing), and an array of slots 606 into which cartridges can be loaded. As shown in FIG. 6, the slots included in the array 606 may be arranged linearly in parallel so that more than one cartridge can be loaded into the tray 602 at one time.

Each cartridge 608 may include a probe well 610 and a protective cap 612 that is designed to accommodate a probe when loaded into the probe well 610. At a high level, the probe well 610 may serve as a protective container for the probe. In some embodiments, the protective cap 612 is mechanically coupled to the probe well 610, for example, via a hinge. After a probe is inserted into the probe well 610, the protective cap 612 can be folded over the probe well 610 to partially or fully enclose the probe. The probe may be vertically stored inside the probe well 610 such that its distal end is suspended in the probe well 610 when the protective cap 612 is in the closed position.

Each cartridge 608 may comprise two separate compartments of wells. The first compartment may include a sample well 614, a first reagent well 616, and a second reagent well 618. The first and second reagent wells 616, 618 may contain the same reagent, or the first and second reagent wells 616, 618 may contain different reagents. The second compartment may include one or more reconstitution wells 620, one or more wash wells 622, and a measurement well 624. The reconstitution well(s) 620 may contain a reconstitution buffer to be dispensed into the sample contained in the sample well 614, as well as the first and second reagent wells 616, 618 for reconstituting dry reagents in these wells. The fluids contained in the reconstitution well(s) 620 may be transferred to the sample well 614, first reagent well 616, or second reagent well 618 using pipettes, for example. Each wash well 622 may contain an aqueous solution to wash the probe after placing the probe in the sample well 614, first reagent well 616, or second reagent well 618.

Meanwhile, the measurement well 624 may contain another aqueous solution. In some embodiments. the aqueous solution in the measurement well 624 is the same as the aqueous solution in the well well(s) 622. In other embodiments, the aqueous solution in the measurement well 624 is different than the aqueous solution in the well well(s) 622. In some embodiments, the measurement well 624 has a light-transmissive bottom that can be used for optical reading of the biolayer that is formed along the distal end of the probe during a biochemical test. For example, the measurement well 624 may have a light-transmissive bottom if the robotic cart is designed for fluorescent assays, chemiluminescent assays, or combination assays (e.g., chemiluminescent-fluorescent assays) rather than interferometric assays. Thus, light may be emitted toward the distal end of the probe through the light-transmissive bottom of the measurement well 624 rather than proximal end of the probe in embodiments where the light is intended to excite fluorescent labels or chemiluminescent labels. In such a scenario, the fluorescent or chemiluminescent signals can be “read” through the light-transmissive bottom of the measurement well 624.

### Overview of Robotic Cart

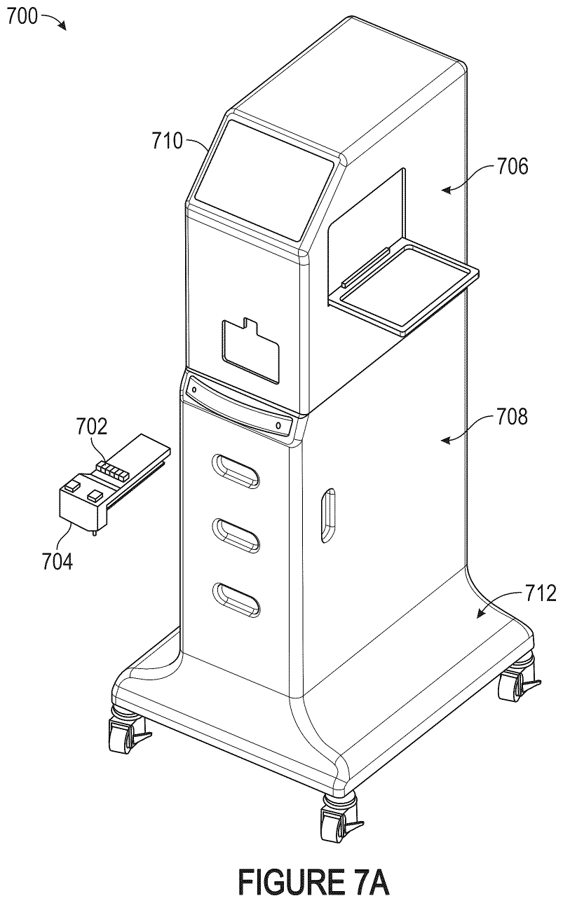

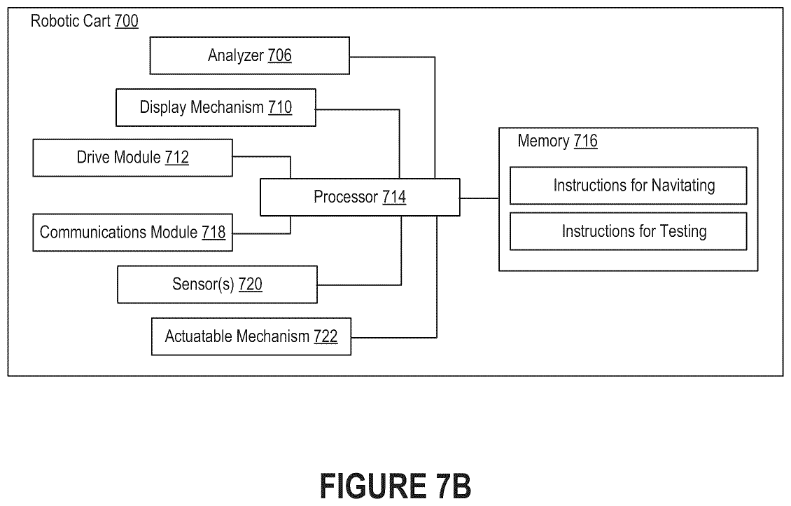

FIG. 7A includes a perspective view of a robotic cart 700 in accordance with various embodiments. FIG. 7B includes a block diagram illustrating various components of the robotic cart 700 shown in FIG. 7A. As discussed above, an operator may deposit a probe in a well of a cartridge 702 where the well contains a sample to be analyzed. While the cartridge 702 could be loaded into the robotic cart 700 on its own, the cartridge 702 could also be installed into a tray 704 that could then be loaded into the robotic cart 700. Collectively, the tray 704 and any cartridges installed therein may be referred to as a “cartridge assembly.” In some embodiments, the operator may even be permitted to load the sample (e.g., in a tube) directly into the robotic cart 700.

As shown in FIG. 7A, a cartridge assembly could be loaded through an aperture (e.g., a slot with a movable cover) in the durable housing of the robotic cart 700. The aperture could be a slot with a cover that is manually movable by the operator. Alternatively, the aperture could be a slot with a cover that is automatically movable by the robotic cart 700. For instance, the robotic cart 700 may retract or pivot the cover to expose the slot in response to a determination that the operator is interested in loading the tray 704. This determination could be based on input provided by the operator (e.g., a selection of a digital element shown on a display mechanism 710, a selection of a physical button accessible along the surface of the durable housing), or this determination could be based on an action performed by the operator. As an example, the robotic cart 700 may include circuitry that is able to detect and then examine electronic signatures emitted by nearby beacons. Accordingly, if a tray 704 that includes a beacon is presented to the robotic cart 700 (e.g., positioned near the cover), the robotic cart may be able to detect the electronic signature and then retract or pivot the cover to expose the slot.

Generally, the robotic cart 700 is designed such that when a cartridge 702 is loaded into the robotic cart 700, its contents can be examined by an analyzer 706 located inside the robotic cart 700. The term “analyzer” may collectively refer to the hardware, firmware, or software that is needed to analyze the sample contained in the cartridge. As mentioned above, the analyzer 706 may be able to facilitate interferometric assays, fluorescent assays, chemiluminescent assays, combination assays (e.g., chemiluminescent-fluorescent assays), or any combination thereof. Accordingly, its components may depend on the type(s) of assays to be permitted by the robotic cart 700. As an example, the analyzer 706 may include a light source (e.g., light source 102 of FIG. 1), detector (e.g., detector 104 of FIG. 1), and waveguide (e.g., waveguide 106 of FIG. 1) that are installed within the robotic cart 700 such that when the cartridge 702 is loaded into the robotic cart 700 with a probe installed in the measurement well (e.g., measurement well 624 of FIG. 6), the waveguide is aligned with the probe.

Generally, the operator may be expected to perform aspects of the biochemical test, such as moving the probe between the various wells of the cartridge 702 before the cartridge 702—with the probe loaded therein—into the robotic cart 700 for analysis. However, in some embodiments, the robotic cart 700 includes an actuatable assembly 722 that is designed to move the probe on behalf of an operator. In embodiments where the robotic cart 700 includes the actuatable assembly 722, the operator may simply load the cartridge 702 into the robotic cart 700, and the actuatable assembly 722 may be responsible for removing the probe between the probe well (e.g., probe well 610 of FIG. 6), sample well (e.g., sample well 614 of FIG. 6), first reagent well (e.g., first reagent well 616 of FIG. 6), second reagent well (e.g., second reagent well 618 of FIG. 6), reconstitution wells (e.g., reconstitution wells 620 of FIG. 6), wash wells (e.g., wash wells 622 of FIG. 6), and measurement well (e.g., measurement well 624 of FIG. 6). As an example, the actuatable assembly 722 may remove the probe from the probe well and then load the probe into the sample well, a first wash well, the first reagent well, a second wash well, and the measurement well in succession. As another example, the actuatable assembly 722 may remove the probe from the probe well and then load the probe into the sample well, the first reagent well, a first wash well, the second reagent well, a second wash well, and the measurement well in succession. The order and count of wells into which the probe is loaded may depend on the nature of the biochemical test being performed (and more specifically, the nature of the analyte molecule of interest). In some embodiments the actuatable assembly 722 is able to remove the protective cap (e.g., protective cap 612 of FIG. 6) from the probe well on its own, while in other embodiments the operator is required to remove the protective cap before the cartridge 702 is loaded into the robotic cart 700. At a high level, the actuatable assembly 722 may include a grasping element, such as actuatable pair of elements or actuatable manipulator (also referred to as an “arm”), that is able to grasp and move the probe. In operation, the grasping element could grasp the probe near its proximal end, or the grasping element could grasp the probe near its midpoint so as to avoid potentially damaging (e.g., scratching) its proximal end.

Assume, for example, that an operator is interested in conducting a biochemical test to determine whether a given analyte is present in a sample. In such a scenario, the operator may provide, to a control platform, input indicative of a request to summon the robotic cart 700. Upon receiving this input, the control platform may transmit an instruction to the robotic cart 700 to locate itself proximate to the operator. In some embodiments the operator may be prompted to specify her location (e.g., through a computing device used to interface with the control platform), while in other embodiments the location of the operator may be inferred by the control platform (e.g., based on a location of the computing device, a schedule of the operator, etc.).

The robotic cart 700 can then autonomously navigate its physical environment to reach the intended destination. To accomplish this, the robotic cart 700 may rely on outputs generated by one or more sensors 720 installed therein. These sensors may include proximity sensors, ambient light sensors, and the like. For example, the robotic cart 700 may include one or more light detection and ranging (LiDAR) sensors that use pulsed lasers to detect the variable distance between the robotic cart 700 and obstacles in the physical environment.

After the robotic cart 700 arrives at its intended destination, the operator may open the storage 708 and remove a cartridge 702. Normally, ancillary materials like syringes and needles will already be readily accessible, through the robotic cart could also store these materials in the storage 708. The operator can obtain a sample and deposit the sample into the cartridge 702, and then the operator can load the cartridge 702 into the robotic cart 700 for analysis. The outputs that are produced by the robotic cart 700 (and, more specifically, its analyzer 706) may be presented by the robotic cart.

For example, these outputs—or analyses of these outputs—may be shown on a display mechanism 710. The display mechanism 710 can be any component that is operable to visually convey information. For example, the display mechanism 710 may be a panel that includes LEDs, organic LEDs, liquid crystal elements, or electrophoretic elements. In some embodiments, the display mechanism 710 is touch sensitive. Thus, an operator may be able to provide input to the robotic cart 700 by interacting with the display mechanism 710. In embodiments where the display mechanism 710 is not touch sensitive, the operator may be able to interact with the robotic cart 700 using a control device, such as a keyboard, a physical element (e.g., a mechanical button), or a pointing device (e.g., a computer mouse). Additionally or alternatively, the operator may be able to interact with the robotic cart 700 using another computing device that is communicatively connected to the robotic cart 700. For example, the operator may provide instructions to the robotic cart 700 via the control platform that is responsible for managing the robotic cart 700.

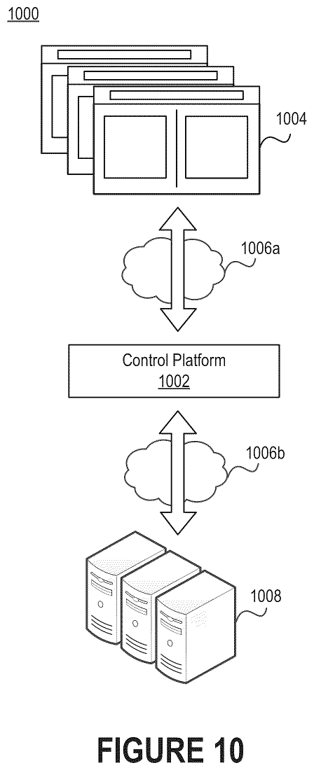

Outputs that are produced by the robotic cart 700 may be stored in its memory 716. The outputs that are produced by the robotic cart 700 can also be forwarded to the control platform that is responsible for managing the robotic cart 700. Accordingly, the outputs may not only be viewable on another computing device that is communicatively connected to the robotic cart 700, either directly or indirectly (e.g., via a network-accessible server system as discussed below with reference to FIG. 10), but the outputs may also be stored on the other computing device. This helps ensure that outputs are readily accessible in the event that the robotic cart 700 is no longer operable (e.g., experiences a power failure or an equipment failure). Storing the outputs in multiple locations may increase the likelihood of authorized access. Therefore, the robotic cart 700 may be designed to encrypt its outputs for safekeeping purposes. For example, the robotic cart 700 (and more specifically, its processor 714) may employ an encryption scheme that encrypts outputs with a cryptographic key. Outputs may be forwarded to the control platform in encrypted form, but the control platform may be able to decrypt the outputs using a complementary cryptographic key.

In some embodiments, the robotic cart 700 performs actions based on the current state of inventory within the storage 708. As an example, when the number of unused cartridges in the storage 708 falls beneath a threshold (e.g., 3 or 5), the robotic cart 700 may transmit a notification to the control platform that is representative of a request for additional supply of unused cartridges. As another example, when the number of used cartridges loaded into the robotic cart 700 exceeds a threshold (e.g., 5 or 7), the robotic cart 700 may transmit a notification to the control platform that is representative of a request to clean or service the robotic cart 700. Thus, the robotic cart 700 may be able to consistently and intelligently monitor its state, though decisions on which actions to perform may be made by the control platform.

The robotic cart 700 may include a drive module 712 that allows it to autonomously navigate a physical environment. The drive module 712 (also referred to as a “movement module”) may include multiple controllable wheels that enable the robotic cart 700 to follow a safe path through the physical environment as determined based on outputs produced by the sensors 720 mentioned above. These wheels may be collectively and/or individually controllable by a processor 714. In embodiments where these wheels are individually controllable, the processor 714 may be configured to generate, based on the data produced by the sensors 720, a separate signal for each of these wheels, so as to autonomously avoid the obstacles and navigate the ambient environment. These signals may not be identical, however. For example, the robotic cart 700 may move through the use of differential steering, in which wheels are rotated at different speeds to change direction. Alternatively, only a subset of these wheels may be “drive” via signals output by the processor 714, and therefore at least some of the wheels may be freely manipulable.

As shown in FIG. 7A, the wheels can be coupled to the underside of the durable housing of the robotic cart 700. The illustrated wheels are casters that are able to rotate and swivel relative to the drive module 712 during movement along a surface. Normally, all of the wheels are powered and thus steerable. However, a subset of the wheels may be powered in some embodiments. For example, only the wheels located along the rear side of the durable housing may be powered in order to conserve power. In such embodiments, the remaining non-powered wheels may be non-steerable or steerable. The robotic cart 700 preferably includes 3, 4, or 6 wheels to provide sufficient stability as it moves, though embodiments could include any number of wheels.

As shown in FIG. 7B, the robotic cart 700 can include a processor 714, memory 716, and communication module 718 in addition to the analyzer 706, display mechanism 710, drive module 712, and sensor(s) 720 discussed above. Each of these components is discussed in greater detail below.

The processor 714 can have generic characteristics similar to general-purpose processors, or the processor 714 may be an application-specific integrated circuit (ASIC) that provides control functions to the robotic cart 700. The processor 714 can be connected to all components of the robotic cart 700, either directly or indirectly, for communication purposes.

The memory 716 may be comprised of any suitable type of storage medium, such as static random-access memory (SRAM), dynamic random-access memory (DRAM), electrically erasable programmable read-only memory (EEPROM), flash memory, or registers. In addition to storing instructions (e.g., instructions for controlling the drive system 712 for navigation purposes, instructions for controlling the analyzer 706 for testing purposes) that can be executed by the processor 714, the memory 716 can also store data that is generated by the various components of the robotic cart 700. For example, over the course of a biochemical test, the analyzer 706 may generate values that relate to development of a biolayer as discussed above with reference to FIGS. 1-5. These values may be stored in the memory 716 in a “raw” form, or these values may be stored in the memory 716 in a “processed” form, for example, following processing by the processor 714.

The communication module 718 may be responsible for managing communications between the various components of the robotic cart 700, or the communication module 718 may be responsible for managing communications with other computing devices. For example, in embodiments where the control platform that manages the robotic cart 700 is executed by another computing device, the communication module 718 can facilitate communications with the other computing device. The communication module 718 may be wireless communication circuitry that is designed to establish communication channels with other computing devices. Examples of wireless communication circuitry include antenna modules configured for cellular networks (also referred to as “mobile networks”) and integrated circuits configured for Near Field Communication (NFC), Wireless Universal Serial Bus (USB), Bluetooth, and the like.

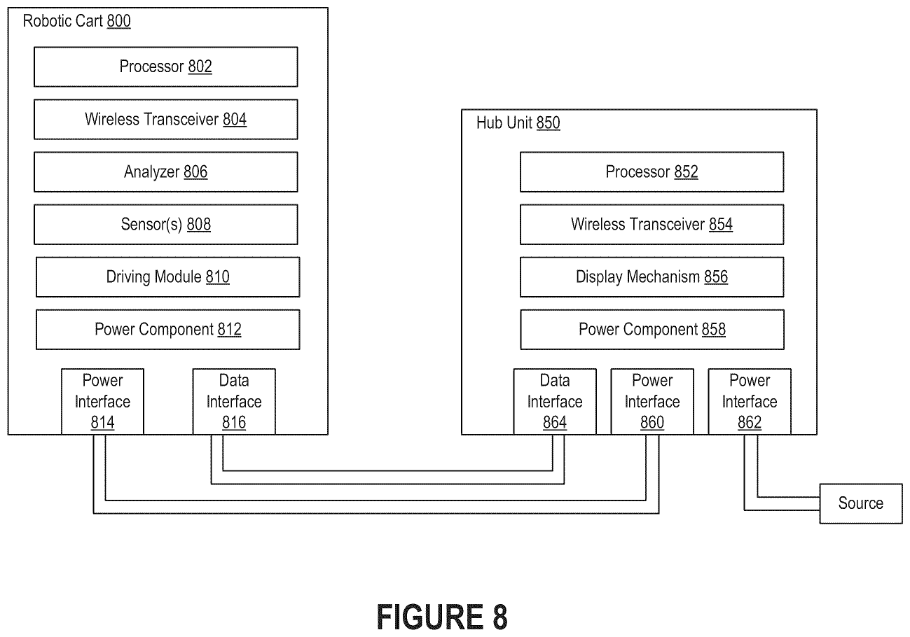

FIG. 8 is a high-level block diagram illustrating how a robotic cart 800 may be able to interface with a hub unit 850, for example, for charging purposes. In some embodiments, the hub unit 850 (also referred to as a “docking station” or “charging station”) is associated with a single robotic cart 800. In other embodiments, the hub unit 850 is associated with multiple robotic carts, and those robotic carts may be able to interface with the hub unit 850 either sequentially or simultaneously (e.g., in embodiments where the hub unit 850 includes multiple docking mechanisms).

As mentioned above, the robotic cart 800 may be able to autonomously traverse a physical environment to situate itself near an operator who is interested in conducting a biochemical test. The robotic cart 800 may include a processor 802, a wireless transceiver 804, an analyzer 806, one or more sensors 808, a drive module 810, and a power component 812 that is electrically coupled to a power interface 814. These components may reside within a durable housing (also referred to as the “structural body” of the robotic cart 800). Embodiments of the robotic cart 800 can include any subset of the components shown in FIG. 8, as well as additional components not illustrated here.

The processor 802 may be responsible for controlling the drive module 810 to navigate the physical environment. Assume, for example, that the robotic cart 800 receives, via the wireless transceiver 804, input indicative of a request to navigate to a given location. In such a scenario, the robotic cart 800 can control the drive module 810 based on outputs produced the sensor(s) 808 to travel from its original location to the given location. The sensor(s) 808 may include proximity sensors, ambient light sensors, and the like. Generally, the request to navigate to the given location is submitted by an operator who is interested in performing a biochemical test. Accordingly, after the robotic cart 800 arrives at the given location, the processor 802 may control the analyzer 806 to administer, conduct, manage, or otherwise support the biochemical test.

Over the course of the biochemical test, data generated by the analyzer 806 may be stored, at least temporarily, in a memory (not shown). In some embodiments, the processor 802 are responsible for processing and then examining the data so as perform analysis locally. In other embodiments, the data is forwarded onward to a destination for analysis. For example, the processor 802 may process the data prior to transmission downstream to the hub unit 850 or another computing device.

In some embodiments, the robotic cart 800 and the hub unit 850 transmit data between one another via a physical connection between respective data interfaces 816, 864. For example, when the robotic cart 800 physically docks with the hub unit 850, data generated by the analyzer 806 may be forwarded to the data interface 816 of the robotic cart 800 for transmission to the data interface 864 of the hub unit 800. Alternatively, the data interface 816 may be part of the wireless transceiver 804. In such embodiments, the wireless transceiver 804 could be configured to establish a wireless connection with a wireless transceiver 854 of the hub unit 850. The wireless transceivers 804, 854 may communicate with one another via a short-range communication protocol (e.g., NFC, Wireless USB, or Bluetooth), a cellular data protocol (e.g., LTE, 4G, or 5G), or a proprietary point-to-point protocol.

Because the robotic cart 800 is autonomous, its power component 812 may become depleted over time. At a high level, the power component 812 may be responsible for providing power to the other components of the robotic cart 800 as necessary. Examples of power components include rechargeable lithium-ion (Li-Ion) batteries, rechargeable nickel-metal hydride (NiMH) batteries, rechargeable nickel-cadmium (NiCad) batteries, etc. By interfacing with the hub unit 850, the robotic cart 800 may be able to recharge its power component 812. For example, when the robotic cart 800 physically docks with the hub unit 850, power may be transmitted form the hub unit 850 to the robotic cart 800 via a physical connection of electrical contacts included in respective power interfaces 814, 860. Accordingly, power may automatically be transferred from the hub unit 850 to the robotic cart 800 when the robotic cart 800 is determined to be properly docked with the hub unit 850 (e.g., based on whether there is a physical connection between the electrical contacts). Note that the hub unit 850 may include a processor 852 that may manage power transfer, among other things. For example, if the processor 852 determines that the robotic cart 800 is fully charged, then the processor 852 may terminate power transfer from the hub unit 850. Moreover, the hub unit 850 may include a display mechanism 816 that is able to indicate whether the robotic cart 800 is properly docked with the hub unit 850, whether power is presently being transferred to the robotic cart 800, whether data is presently being transferred from the robotic cart 800, etc.

As shown in FIG. 8, the hub unit 850 may also include another power interface 862 that allows the hub unit 850 to be physically connected to a power source (e.g., an electrical outlet) from which power can be obtained without limitation. Alternatively, the power interface 862 may be representative of an integrated circuit that is able to wirelessly receive power from the power source. The integrated circuit may be able to receive power transmitted in accordance with the Qi standard developed by the Wireless Power Consortium or some other wireless power standard. Similarly, the power interfaces 814, 860 of the robotic cart 800 and hub unit 850 could be integrated circuits that are able to wirelessly transfer power.

Regardless of its power, the power interface 862 may allow power to be received from a source external to the hub unit 850. As mentioned above, the hub unit 850 may also include a power component 858 that can store power received at the power interface 862. However, the power component 858 may not be present in some embodiments. Instead, power received by the hub unit 850 at the power interface 862 from the source may simply be directed to the other power interface 860 for transmission onward to the robotic cart 800, when appropriate.

As part of its autonomous nature, the robotic cart 800 may be configured to intelligently charge itself when not in use. For example, if the robotic cart 800 has not moved for a predetermined interval of time (e.g., due to a lack of requests from operators), then the robotic cart 800 may return to its corresponding hub unit 850 that serves as a charging station. Power may automatically flow from the hub unit 850 to the robotic cart 800 via wired transmission of power when the robotic cart 800 is physically docked to the hub unit 850. Likewise, power may automatically flow from the hub unit 850 to the robotic cart 800 via wireless transmission of power when the robotic cart 800 is physically docked to, or simply located near, the hub unit 850.

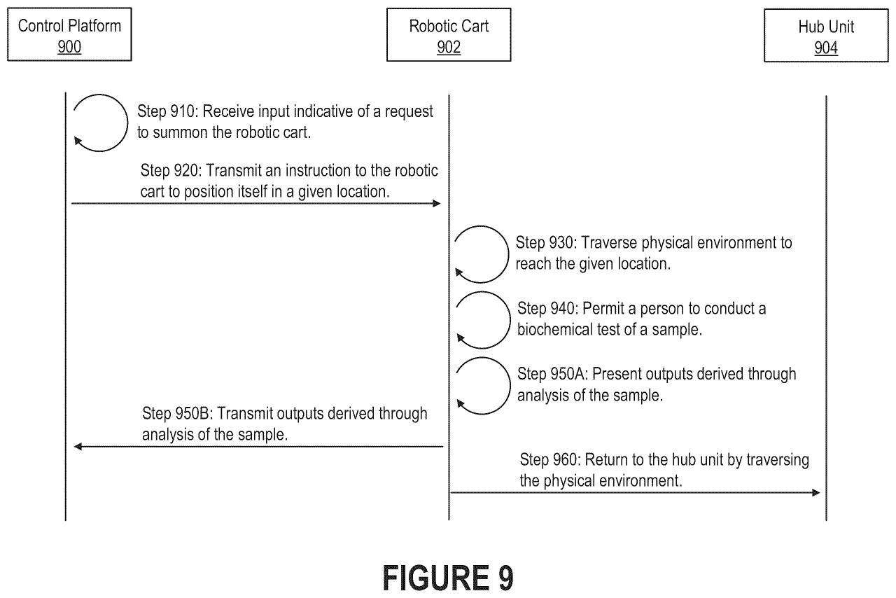

FIG. 9 includes a high-level illustration of communications that may occur between a control platform 900, a robotic cart 902, and a hub unit 904. Initially, the control platform 900 may receive input indicative of a request to summon the robotic cart 902 (step 910). As mentioned above, the control platform 900 normally resides on, and thus is executed by, a computing device that is communicatively connected to the robotic cart 902, either directly or indirectly. One example of a computing device is a mobile phone associated with the person who has summoned the robotic cart 902. Another example of a computing device is a network-accessible server system, though in such embodiments the control platform 900 may be accessible via a computer program executing on a mobile phone, tablet computer, mobile workstation, etc.

Then, the control platform 900 can transmit an instruction to the robotic cart 902 to position itself in a given location (step 920). The instruction may depend on how the control platform 900 is implemented. For example, in an embodiment where the control platform 900 resides on a computing device that is locally accessible to the person who is interested in summoning the robotic cart 904, the given location may be established based on the current location of the computing device. Alternatively, the given location may be specified in the request submitted by the person.

Upon receiving the instruction, the robotic cart 902 may autonomously traverse its physical environment to position itself in the given location (step 930). Thereafter, the robotic cart 902 may permit the person to conduct a biochemical test (step 940) as discussed above. For example, the person may retrieve a cartridge from the robotic cart, obtain a sample from a patient, and then deposit the sample into the cartridge before loading the cartridge into the robotic cart 902 for analysis. Generally, the robotic cart 902 will analyze the sample while situated in the given location. However, the robotic cart 902 may return to its corresponding hub unit 904 before analyzing the sample in some situations (e.g., where insufficient power is available to complete the biochemical test). As shown in FIG. 9, the outputs that are produced by the robotic cart from analyzing the sample can be presented for display (step 950A) or transmitted to the control platform (step 950B).

After the biochemical test is complete, the robotic cart 902 may return to its corresponding hub unit 904 (step 960). In some embodiments, this is done automatically. For example, the robotic cart 902 may automatically traverse the physical environment to dock with the hub unit 904 upon determining that the robotic cart 902 is no longer of use to the person (e.g., based on inactivity). As another example, the robotic cart 902 may return to dock with the hub unit 904 when the power stored in its power component falls beneath a predetermined threshold (e.g., 20, 25, or 30 percent of total capacity). In other embodiments, the robotic cart 904 returns to the hub unit 904 upon receiving a request to do so. For example, the person may be prompted to indicate when she is done with the robotic cart 904, and when such an indication is made, the robotic cart 904 may return to the hub unit 904.

### Overview of Control Platform

As mentioned above, a control platform may be responsible for managing movement of the robotic cart throughout its physical environment, among other things. FIG. 10 illustrates a network environment 1000 that includes a control platform 1002. Individuals can interact with the control platform 1000 via interfaces 1004. For example, an operator may be able to summon a robotic cart via an interface, as well as instruct the robotic cart to travel to another location (e.g., its default location). As another example, the operator may be able to view the status of a robotic cart via an interface. The interface may indicate the current location of the robotic cart, the current power level of the robotic cart, the supplies currently stored in the robotic cart, etc. Thus, the interfaces 1004 may serve as interactive dashboards through which operators can manage robotic carts. Additionally or alternatively, the interfaces 1004 may serve as informative dashboards through which operators and other individuals can observe information regarding robotic carts. For example, operators may be able to review analyses of data generated by an analyzer included in a robotic cart as part of a biochemical test.

As shown in FIG. 10, the control platform 1002 may reside in a network environment 1000. The control platform 1002 may reside on, and be executed by, a computing device (not shown) that is connected to one or more networks 1006a-b. The computing device could be a robotic cart or another device, such as a mobile phone, tablet computer, computer server, etc. The networks 1006a-b can include personal area networks (PANs), local area networks (LANs), wide area networks (WANs), metropolitan area networks (MANs), cellular networks, the Internet, etc. Additionally or alternatively, the control platform 1002 can be communicatively coupled to one or more computing devices over a short-range wireless connectivity technology, such as Bluetooth, NFC, Wireless USB, and the like.

The interfaces 1004 may be accessible via a web browser, mobile application, desktop application, over-the-top (OTT) application, or some other computer program.

For example, an operator may be able to initiate a mobile application that is executing on her mobile phone and then input, through an interface generated by the mobile application, data that is representative of a request to summon a robotic cart to her current location. The operator may specify her current location through the interface, or her current location may be inferred by the control platform 1002 based on, for example, the strength or presence of wireless access point (WAP) signals received by the mobile phone, the strength or presence of beacons (e.g., Bluetooth beacons or WiFi beacons), the strength or presence of background noises, and the like. Alternatively, the control platform 1002 may infer her current location based on a schedule that is associated with the operator. If, for example, the schedule indicates that the operator is presently scheduled to be in a given room, then the control platform 1002 may infer that the operator is located in the given room.

As another example, an operator may be able to access an interface on which information regarding a patient can be reviewed through a desktop application that is executing on a mobile workstation. Such information can include name, date of birth, diagnoses, symptoms, or medications. With this information, the healthcare professional may be able to readily establish the status of the patient, render diagnoses, and the like. Through this interface, the operator may be able to manage a robotic cart as discussed above.

Accordingly, the interfaces 1004 may be viewed on computing devices such as mobile workstations (also referred to as “medical carts”), personal computers, tablet computers, mobile phones, wearable electronic devices, and the like.

In some embodiments, at least some components of the control platform 1002 are hosted locally. That is, part of the control platform 1002 may reside on the computing device used to access one of the interfaces 1004. For example, the control platform 1002 may be embodied as a mobile application executing on a mobile phone associated with an operator, or the control platform 1002 may be embodied as a desktop application executing on a mobile workstation associated with a healthcare facility. Note, however, that the mobile and desktop applications may be communicatively connected to a network-accessible server system 1008 on which other components of the control platform 1002 are hosted. As mentioned above, components of the control platform 1002 could also be hosted on the robotic cart itself. Thus, the control platform 1002 could be distributed across, or at least communicatively connected to, the robotic cart, network-accessible server system 1008, and computing device through which input is provided to the control platform 1002.

In other embodiments, the control platform 1002 is executed entirely by a cloud computing service operated by, for example, Amazon Web Services®, Google Cloud Platform™, or Microsoft Azure®. In such embodiments, the control platform 1002 may reside on a network-accessible server system 1008 that comprises one or more computer servers. These computer servers can include computer-implemented models, algorithms (e.g., for processing data generated by the analyzer of the robotic cart), patient information (e.g., profiles, credentials, and health-related information such as age, date of birth, geographical location, disease classification, disease state, healthcare provider, etc.), and other assets. Those skilled in the art will recognize that this information could also be distributed among the network-accessible server system 1008 and one or more computing devices.

### Processing System

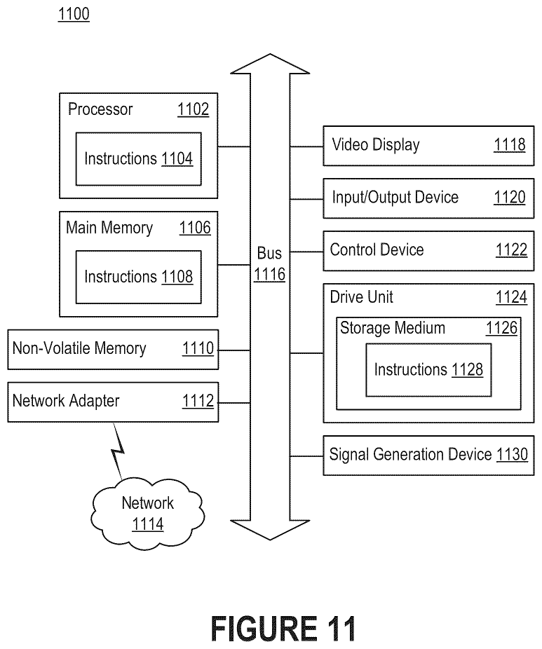

FIG. 11 is a block diagram illustrating an example of a processing system 1100 in which at least some operations described herein can be implemented. For example, components of the processing system 1100 may be hosted on a robotic cart (e.g., robotic cart 700 of FIGS. 7A-B, robotic cart 800 of FIG. 8, or robotic cart 902 of FIG. 9). As another example, components of the processing system 1100 may be hosted on a hub unit (e.g., hub unit 850 of FIG. 8 or hub unit 904 of FIG. 9). As another example, components of the processing system 1100 may be hosted on a computing device that is used to interface with a control platform (e.g., control platform 1000 of FIG. 10).

The processing system 1100 may include a processor 1102, main memory 1106, non-volatile memory 1110, network adapter 1112, video display 1118, input/output device 1120, control device 1122, drive unit 1124 that includes a storage medium 1126, or signal generation device 1130 that are communicatively connected to a bus 1116. The bus 1116 is illustrated as an abstraction that represents one or more physical buses or point-to-point connections that are connected by appropriate bridges, adapters, or controllers. The bus 1116, therefore, can include a system bus, Peripheral Component Interconnect (PCI) bus, PCI-Express bus, HyperTransport bus, Industry Standard Architecture (ISA) bus, Small Computer System Interface (SCSI) bus, Universal Serial Bus (USB), Inter-Integrated Circuit (I²C) bus, or a bus compliant with Institute of Electrical and Electronics Engineers (IEEE) Standard 1394.

The processing system 1100 may share a similar computer processor architecture as that of a computer server, personal computer, tablet computer, mobile phone, wearable electronic device (e.g., an activity tracker or head-mounted display), network-connected electronic device (e.g., a television or home assistant device), or another computing device that is capable of executing a set of instructions (sequential or otherwise) that specify actions to be taken by the processing system 1100.

While the main memory 1106, non-volatile memory 1110, and storage medium 1126 are shown to be a single medium, the terms “storage medium” and “machine-readable medium” should be taken to include a single medium or multiple media that stores instructions 1128. The terms “storage medium” and “machine-readable medium” should also be taken to include any medium that is capable of storing, encoding, or carrying instructions for execution by the processing system 1100.

In general, the routines executed to implement the embodiments of the present disclosure may be implemented as part of an operating system or a specific component, program, object, module, or sequence of instructions (collectively referred to as “computer programs”). The computer programs typically comprise instructions (e.g., instructions 1104, 1108, 1128) set at various times in various memories and storage devices in a computing device. When read and executed by the processor 1102, the instructions may cause the processing system 1100 to perform operations to execute various aspects of the present disclosure.

While embodiments have been described in the context of fully functioning computing devices, those skilled in the art will appreciate that the embodiments are capable of being distributed in a variety of forms. The present disclosure applies regardless of the particular type of machine- or computer-readable medium used to actually cause the distribution. Further examples of machine- and computer-readable media include recordable-type media such as volatile memory, non-volatile memory 1110, removable disks, hard disk drives, optical disks (e.g., Compact Disk Read-Only Memory (CD-ROMS) and Digital Versatile Disks (DVDs)), cloud-based storage media, and transmission-type storage media such as digital and analog communication links.

The network adapter 1112 enables the processing system 1100 to mediate data in a network 1114 with an entity that is external to the processing system 1100 through any communication protocol supported by the processing system 1100 and the external entity. The network adapter 1112 can include a wired network adaptor card, wireless network interface card, switch, protocol converter, gateway, bridge, hub, receiver, repeater, or transceiver that includes an appropriate chip (e.g., enabling communication over Bluetooth or Wi-Fi).

### REMARKS

The foregoing description of various embodiments of the technology has been provided for the purposes of illustration and description. It is not intended to be exhaustive or to limit the claimed subject matter to the precise forms disclosed.

Many modifications and variation will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. A robotic cart for facilitating biochemical tests, the robotic cart comprising:
a durable housing that includes an aperture adapted to receive a cartridge containing a sample from a patient;
a test system that is configured to perform a biochemical test on the sample and provide test results;
a sensor that is configured to produce data indicative of obstacles in an ambient environment;
a drive module that includes multiple independently controllable wheels; and
a processor that is configured to generate, based on the data produced by the sensor, multiple signals for the multiple independently controllable wheels of the drive module, so as to autonomously avoid the obstacles and navigate the ambient environment.

2. The robotic cart of claim 1, wherein the sensor is a light detection and ranging (LiDAR) sensor that uses one or more pulsed lasers to detect a variable distance between the robotic cart and the obstacles in the ambient environment.

3. The robotic cart of claim 1, wherein the aperture is a slot that is designed to receive the cartridge and has a movable cover.

4. The robotic cart of claim 1, wherein the test system includes
(i) a light source that, in operation, emits light toward a probe included in the cartridge, and
(ii) a detector that, in operation, records a spectrum of the light reflected by the probe.

5. The robotic cart of claim 4, wherein the light emitted by the light source is conveyed to the probe via a waveguide, and wherein the light received from the probe is conveyed to the detector via the waveguide.

6. The robotic cart of claim 4, wherein the detector is able to record intensity of the light received from the probe at a plurality of wavelengths.

7. The robotic cart of claim 4, further comprising:
an actuatable assembly that, in operation, moves a probe amongst different wells included in the cartridge.

8. A diagnostic system comprising:
a cartridge that includes a plurality of wells, at least one of which includes a sample from a patient is deposited by an operator;
a robotic cart into which the cartridge is insertable by the operator for analysis; and
a control platform that is responsible for managing
(i) movement of the robotic cart through a physical environment, and
(ii) testing of the sample when the cartridge is inserted into the robotic cart.

9. The diagnostic system of claim 8, wherein the control platform is executed by a computing device that is communicatively connected to the robotic cart via a network.

10. A method performed by a robotic cart that includes a test system for performing a biochemical test, the method comprising:
receiving, from a control platform, input that is indicative of an instruction to be positioned in a given location inside a facility;
traversing the facility in an autonomous manner based on signals output by one or more sensors included in the robotic cart;
permitting insertion of a cartridge with a sample deposited therein; and
performing a biochemical test with the test system in response to the cartridge being inserted into the robotic cart.

11. The method of claim 10, further comprising:
causing display of an output of the biochemical test on a display mechanism of the robotic cart.

12. The method of claim 10, further comprising:
transmitting an output of the biochemical test to a computing device via a network, for display by the computing device.

13. The method of claim 10, further comprising:
encrypting an output of the biochemical test, and
storing the encrypted output in a memory of the robotic cart.

14. A non-transitory medium with instructions stored thereon that, when executed by a processor of a computing device, cause the computing device to perform operations comprising:
receiving first input that is indicative of a request to summon a robotic cart;
transmitting an instruction to the robotic cart to position itself in a given location;
receiving second input that is indicative of an output derived by the robotic cart through analysis of a sample taken from a patient; and
displaying an analysis of the output on a display mechanism, for review by an operator of the computing device.

15. The non-transitory medium of claim 14, wherein the given location included in the instruction is specified by the operator in the request.

16. The non-transitory medium of claim 14, wherein the given location included in the instruction is inferred based on a current location of the computing device.

17. The non-transitory medium of claim 14, wherein the given location included in the instruction is inferred based on a schedule of the operator.
